(19) United States
(12) Patent Application Publication
(10) Pub. No.: US 2024/0428894 A1
(43) Pub. Date: Dec. 26, 2024

(54) SYSTEMS AND METHODS FOR CHARACTERIZING HYDROCARBON-CONTAINING FLUIDS

(71) Applicant: Schlumberger Technology Corporation, Sugar Land, TX (US)

(72) Inventor: Dariusz Strapoc, Clamart (FR)

(21) Appl. No.: 18/752,345

(22) Filed: Jun. 24, 2024

(30) Foreign Application Priority Data
Jun. 23, 2023 (EP) 23306003.7

(51) Int. Cl. G16C 20/20; G01N 33/28; G16C 20/70

(52) U.S. Cl. CPC G16C 20/20; G01N 33/2823; G16C 20/70

(57) ABSTRACT

A system includes processing circuitry and a non-transitory, computer-readable medium that includes instructions that cause processing circuitry to receive logging data regarding a fluid. The logging data is indicative of a plurality of isotope ratios of a plurality of alkanes of the fluid. The instructions also cause the processing circuitry to determine, based on at least a first isotope ratio of the plurality of isotope ratios of the logging data corresponding to a first alkane of the plurality of alkanes, a thermal maturity of the fluid. Additionally, the instructions cause the processing circuitry to determine, based on at least a second isotope ratio of the plurality of isotope ratios corresponding to a second alkane of the plurality of alkanes, a gas-oil ratio (GOR) of the fluid. Furthermore, the instructions cause the processing circuitry to cause display of the thermal maturity of the fluid and the GOR of the fluid.

### CROSS REFERENCE TO RELATED APPLICATION

This application claims priority to and the benefit of European Patent Application No. 23306003.7, titled “SYSTEMS AND METHODS FOR CHARACTERIZING HYDROCARBON-CONTAINING FLUIDS,” filed Jun. 23, 2023, the entire disclosure of which is hereby incorporated herein by reference.

### BACKGROUND

The present disclosure relates generally to techniques for analyzing logging data, such as advanced mud gas logging data to generate data or determinations regarding fluid type, fluid properties, and fluid alterations of petrochemical samples.

During the drilling process of an oil well or of a well of another effluent—in particular gas, vapor or water—drilling mud may be brought to the surface. An analysis may be performed on the mud gas to generate data regarding properties of gas in the formation, for example, at various depths of the formation. However, such analyses may be inaccurate, for not taking into account conditions or other considerations that may alter the results of such analyses, such as potential fluid alterations, fluid maturity, and mixing. Therefore, it is desirable to have an improved method to analyze hydrocarbon-containing fluids such as mud gas.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

Certain embodiments of the present disclosure include a system that may include processing circuitry and a non-transitory, computer-readable medium that includes instructions that cause processing circuitry to receive logging data regarding a fluid. The logging data is indicative of a plurality of isotope ratios of a plurality of alkanes of the fluid. The instructions may also cause the processing circuitry to determine, based on at least a first isotope ratio of the plurality of isotope ratios of the logging data corresponding to a first alkane of the plurality of alkanes, a thermal maturity of the fluid. Additionally, the instructions may cause the processing circuitry to determine, based on at least a second isotope ratio of the plurality of isotope ratios corresponding to a second alkane of the plurality of alkanes, a gas-oil ratio (GOR) of the fluid. Furthermore, the instructions may cause the processing circuitry to cause display of the thermal maturity of the fluid and the GOR of the fluid.

Certain embodiments of the present disclosure include a non-transitory, computer-readable medium that may include instructions that cause processing circuitry to receive logging data regarding a fluid. The logging data is indicative of a plurality of isotope ratios of a plurality of alkanes of the fluid. Also, the logging data includes a plurality of amounts. Each respective amount of the plurality of amounts corresponds to a respective alkane of the plurality of alkanes. The instructions also cause the processing circuitry to determine, based on at least a first isotope ratio of the plurality of isotope ratios of the logging data corresponding to a first alkane of the plurality of alkanes, a thermal maturity of the fluid. Additionally, the instructions cause the processing circuitry to determine, based on at least a second isotope ratio of the plurality of isotope ratios corresponding to a second alkane of the plurality of alkanes, a gas-oil ratio (GOR) of the fluid. Furthermore, the instructions cause the processing circuitry to determine a gas wetness ratio of the fluid based on at least a portion of the plurality of amounts, determine a fluid typing of the fluid based on the gas wetness ratio and the first isotope ratio, and cause display of the thermal maturity of the fluid, the fluid typing of the fluid, and the GOR of the fluid.

Certain embodiments of the present disclosure include a computer-implemented method that may include receiving, via processing circuitry, logging data regarding a fluid. The logging data is indicative of a plurality of isotope ratios of a plurality of alkanes of the fluid. The computer-implemented method may also include determining, via the processing circuitry and based on at least a first isotope ratio of the plurality of isotope ratios of the logging data corresponding to a first alkane of the plurality of alkanes, a thermal maturity of the fluid. Additionally, the computer-implemented method may include determining, via the processing circuitry and based on at least a second isotope ratio of the plurality of isotope ratios corresponding to a second alkane of the plurality of alkanes, a gas-oil ratio (GOR) of the fluid. Furthermore, the computer-implemented method may include causing, via the processing circuitry, display of the thermal maturity of the fluid and the GOR of the fluid.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 is a schematic diagram of a rotary drilling rig system, in accordance with aspects of the present disclosure;

FIG. 2 is a flowchart for characterizing fluids obtained from subterranean formations, in accordance with aspects of the present disclosure;

FIG. 3 is a graph of isotope ratio of methane (δ¹³C-C₁) versus vitrinite reflectance equivalent (VRE), in accordance with aspects of the present disclosure;

FIG. 4 is a graph of isotope ratio of ethane (δ¹³C-C₂) versus VRE, in accordance with aspects of the present disclosure;

FIG. 5 is a graph of δ¹³C-C₂ versus gas-oil ratio (GOR), in accordance with aspects of the present disclosure;

FIG. 6 is a graph plotting gas wetness ratio versus δ¹³C-C₁ as well as providing regions regarding potential characterizations of fluid samples based on the placement of data points for the samples within the graph, in accordance with aspects of the present disclosure;

FIG. 7 is a chart including several sections with graphs indicative of characterizations of a well or subterranean formation that may be generated by a data analysis system of the rotary drilling rig system of FIG. 1, in accordance with aspects of the present disclosure;

FIG. 8 is a graph plotting gas wetness ratio versus δ¹³C-C₂, in accordance with aspects of the present disclosure; and

FIG. 9 is a graph plotting character versus δ¹³C-C₂, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the terms “connect,” “connection,” “connected,” “in connection with,” and “connecting” are used to mean “in direct connection with” or “in connection with via one or more elements”; and the term “set” is used to mean “one element” or “more than one element.” Further, the terms “couple,” “coupling,” “coupled,” “coupled together,” and “coupled with” are used to mean “directly coupled together” or “coupled together via one or more elements.”

In addition, as used herein, the terms “real-time”, “real-time”, or “substantially real-time” may be used interchangeably and are intended to described operations (e.g., computing operations) that are performed without any human-perceivable interruption between operations. For example, data relating to the systems described herein may be collected, transmitted, and/or used in control computations in “substantially real-time”, such that data readings, data transfers, and/or data processing steps may occur once every second, once every 0.1 second, once every 0.01 second, or even more frequent, during operations of the systems (e.g., while the systems are operating). In addition, as used herein, the terms “automatic” and “automated” are intended to describe operations that are performed are caused to be performed, for example, solely by analysis system without human intervention.

The present disclosure relates to the characterization of fluid or gas (e.g., mud gas) samples. In particular, the presently described techniques enable the automated conversion of advanced mud gas logging data into information about fluid type, fluid properties, and fluid alterations. Specifically, δ¹³C values of gaseous alkanes can be utilized to determine thermal maturity (e.g., scaled to vitrinite reflectance equivalent (VRE)) and gas-oil ratio (GOR).

In stable isotope geochemistry, stable carbon isotopes may be expressed as ratio of ¹³C to ¹²C in an analyte with respect to international reference material. For example, such an isotope ratio (e.g., a δ¹³C value) for methane (C₁) can be expressed as δ¹³C-C₁. δ¹³C values of gaseous alkanes may be used in interpretation of fluid maturity and fluid typing and fluid alterations. Expected ranges of δ¹³C values in nature typically range from −80 to −20‰ or permille for methane, −45 to −20‰ for ethane (C₂), and for −45 to 0‰ for propane (C₃). While in general, the δ¹³C values increase with increasing thermal maturity, alteration processes can impact values. For example, biodegradation generates biogenic methane, which has negative values, typically below −60‰, while thermogenic methane covers range of approximately −55 to −20‰. Additionally, biodegradation may cause increases in δ¹³C of residual non-biodegraded propane. In many cases, in sedimentary basins, there is background biogenic methane from early sedimentary stage. At a later stage, thermogenic hydrocarbons may incorporate or mix with the biogenic methane. Hence, isotopic analysis of alkanes (e.g., methane, ethane, and propane) may be useful when determining maturity of charge and alteration scenarios, while analysis of methane alone can be insufficient. More specifically, a mature gas recharge can occur into a reservoir with less mature fluid (e.g., black oil), which may impact gas-based interpretation of the reservoir. As discussed herein, logging of δ¹³C of alkanes such as methane, ethane, and propane may enable characterization of active hydrocarbon systems, for example, to determine fluid maturity distribution, gradients, and mixing, differentiating thin bed tanks, and predicting fluid properties.

However, mud gas molecular composition, as opposed to isotopic composition, can be vulnerable to mud gas degassing techniques. Specifically, factors for this vulnerability are: 1) mud gas extraction efficiency (depending on mud type, temperature, and density, which control gas solubility and extractability); 2) mud gas recycling in reused drilling mud; and 3) degasser technology. These factors impact molecular gas ratios, as within the C₁-C₅ range (i.e., methane, ethane, propane, butane, and pentane), alkanes have different properties and can be fractionated. As discussed below, isotope logging data may be utilized, for example, as a proxy to determine properties and characteristics of subterranean fluids (e.g., mud gas). While the presently disclosed techniques may be described below as being performed in conjunction with advanced mud gas logging (AMGL), the presently disclosed techniques may be utilized with any isotope logging systems or techniques.

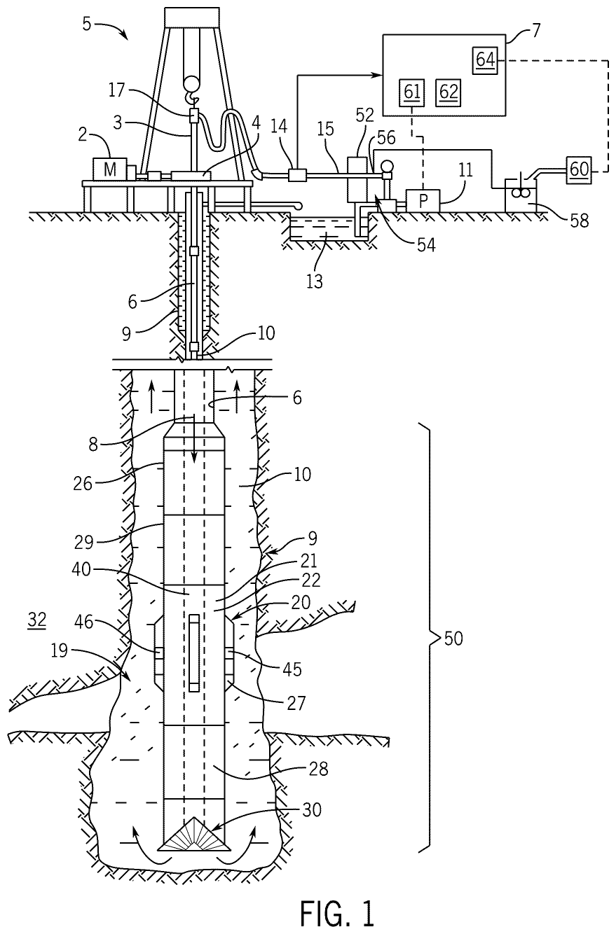

FIG. 1 is a schematic view of an example implementation of a rotary drilling rig system 5. Downhole measurements can be conducted by instruments disposed in a drill collar 20. Such measurements may be stored in memory apparatus of the downhole instruments, or may be telemetered to the surface via conventional measuring-while-drilling (MWD) telemetering apparatus and techniques. For that purpose, an MWD tool sub, schematically illustrated as a tool 29, may receive signals from instruments of the collar 20, and may transmit them via a mud path 8 of a drill string 6 for receipt, e.g., ultimately via a pressure sensor 14 in a stand pipe 15 and/or to other surface instrumentation 7.

The drilling rig system 5 may include a motor 2 that may turn a kelly 3 through the use of a rotary table 4. The drill string 6 may include sections of drill pipe connected end-to-end to the kelly 3 and may be turned thereby. For example, drill collars and/or tools 20, 26, 28, and 29 may be attached to the drilling string 6. Such collars and tools may collectively form a bottom hole assembly (BHA) 50 extending from the drill string 6 to a drilling bit 30. As the drill string 6 and the BHA 50 turn, the drill bit 30 can bore a wellbore 9. An annulus 10 is thus defined between the outside of the drill string 6 (including the BHA 50) and the wellbore 9 through one or more subterranean geological formations 32.

A pump 11 may pump drilling fluid (drilling “mud”) from a source, e.g., from a mud pit 13, via a stand pipe 15, a revolving injector head 17, and the mud path 8 of the kelly 3 and the drill string 6 to the drill bit 30. The mud, which may be a water-based or oil-based drilling mud, may lubricate the drill bit 30 and may carry wellbore cuttings upward to the surface via the annulus 10. If desired, the mud may be returned, for example, to the mud pit 13 or to an appropriate mud regeneration site, where it may be separated from cuttings and the like, degassed, and returned for application again to the drill string 6.

Shale shakers 52 may separate the cuttings from the mud. Once the mud and the cuttings have been separated, the mud and the cutting may be collected and analyzed. Cuttings samples 54 may be collected manually and analyzed in a mud logging cabin with one or more instruments (such as microscope, X-ray fluorescence (XRF), X-Ray Diffraction (XRD), and the like). Alternatively, the cuttings sample may be collected and analyzed automatically at the well site.

The drilling fluid (e.g., drilling mud), may generally sampled at an outlet of the shakers 52 by a sampling device 56 and directed to an extractor 58 that extracts gas from the drilling mud. The gas is then directed to an analyzer 60 in order to detect the content of the gas. In other words, the analyzer 60 may generate logging data (e.g., a data stream) regarding the (mud) gas. The analyzer 60 may include a Thermal Conductivity Detector (TCD), a Flame Ionization Detector (FID), and/or mass spectrometer. The analyzer may also include a gas chromatograph (GC). The analyzer 60 may also be equipment capable of generating log data indicative of the isotopes of carbon (and (relative) amounts of the isotopes) in the mud gas. For example, the analyzer 60 may perform GC-oxidation reactor-isotope ratio mass spectrometry (GC-ox-IRMS). The analyzer 60 may also include a GC-ox-Hollow Wave Guide-Quantum Cascade (HWG-QC) IR laser (GC-ox-HWG-IR) or any other GC-ox-analyzer system. The analyzer 60 may also be or include equipment capable of performing advanced mud gas logging (AMGL), which may involve control on extraction thermodynamics and corrections of extraction efficiency and mud gas recycling biases. As such, the analyzer 60 may be or include any equipment capable of generating isotope logging data regarding gas extracted from the drilling fluid.

It should be noted that the techniques of the present application may be utilized on samples that are otherwise analyzed. For example, isotope lab analysis of mud gas spot samples (e.g., IsoTubes, Vacutainers, gas vials or gas bags) or even bottom hole samples (BHS) solution gas using GC-ox-IRMS may also be employed. Gas molecular composition data can come from any source of alkanes (e.g., C₁-C₅) at the same or similar frequency, such as, mud logging gas chromatography (GC), or performing gas chromatography on the isotope analyzer gas chain, or lab analysis of spot samples. Accordingly, the presently described techniques may be utilized on logging data regardless of whether the logging data is generated onsite (e.g., above ground or down-hole) or offsite (e.g., in a laboratory setting).

The downhole tool (collar) 20 may be any type of downhole tool taking measurement, such as an ultrasonic tool, an electromagnetic or resistivity tool, a sampling tool. For example, the ultrasonic tool may include at least one or more sensors 45, 46, e.g., such as for measuring characteristics of the wellbore 9 and/or fluid, including pressure, standoff, composition, etc. therein during drilling operations. Such measurements may be conducted while the wellbore 9 is being drilled and/or with the drill string 6 and the BHA 50 in the wellbore 9 while the drill bit 30, the BHA 50, and the drill string 6 are not rotating. Such measurements may be conducted while the drill string 6, the BHA 50, and the drill bit 30 are being tripped to and from the bottom of the wellbore 9. The measurements (or data based at least partially thereon) may be transmitted to the surface via the MWD telemetry tool 29 and the internal mud passage 8 of the drill string 6 (or the annulus 10), or they may be recorded and stored downhole and for retrieval at the surface after the drill string 6 and BHA 50 have been removed from the wellbore 9.

The sensors 45, 46 may be mounted on stabilizer fins 27 of the downhole tool 20, as depicted in FIG. 1, or may be mounted in a cylindrical wall 23 of the downhole tool 20. An electronics module 22 may contain electronic circuits, microprocessors, memories, and/or the like, operable to control, and/or to receive, process, and/or store data from the sensors 45, 46, which may be mounted on a sleeve, an inner tube, and/or other section 21 secured around or within the collar of the downhole tool 20. The section 21 and other components of the BHA 50 may include a path 40 by which drilling mud may pass through the interior passage 8 of the drill string 6 to the drill bit 30.

A portion of the drilling rig system 5, such as surface instrumentation 7, may include other sensors for measurement parameters at the surface, such as flow, pressure, weight on bit, torque on bit, etc. and verify that the drilling rig system 5 works properly. As an example, a sensor 61 may be connected to the pump 11 to count the number of strokes of the pump, and a sensor 62 may be present at the kelly 3 or motor to assess the rotations per minute (RPM) or in the weight and torque on bit.

The surface instrumentation 7 may also include data processing system 64 that can encompass one or more, or portions thereof, of the following: control devices and electronics in one or more modules of the BHA 50 (such as a downhole controller), a remote computer system (not shown), communication equipment, and other equipment. The data processing system may include one or more computer systems or devices and/or may be a distributed computer system. For example, collected data or information may be stored, distributed, communicated to a human wellsite operator, and/or processed locally or remotely.

The data processing system 64 may, individually or in combination with other system components, also be linked to all or part of the sensors, downhole or at the surface, to process the measurements and may perform the methods and/or processes described below, or portions thereof. For example, the data processing system 64 may include processor capability for collecting data obtained from the sensors at the surface or downhole. Methods and/or processes within the scope of the present disclosure may be implemented by one or more computer programs that run in a processor located, e.g., in one or more modules of the BHA 50 and/or surface equipment of the drilling rig system 5 and/or a remotely located processing system that is communicatively coupled to the BHA 50 of the drilling rig system 5 (e.g., via a wired or wireless connection). Such programs may utilize data received from the BHA 50 via mud-pulse telemetry and/or other telemetry means, and/or may transmit control signals to operative elements of the BHA 50. The programs may be stored on a tangible, non-transitory, computer-usable storage medium associated with the one or more processors of the BHA 50 and/or surface equipment, such as the surface instrumentation 7, of the drilling rig system 5, or may be stored on an external, tangible, non-transitory, computer-usable storage medium electronically coupled to such processor(s). The storage medium may be one or more known or future-developed storage media, such as a magnetic disk, an optically readable disk, flash memory, or a readable device of another kind, including a remote storage device coupled over a communication link, among other examples. Accordingly, the data processing system 64 may include processing circuitry (e.g., one or more processors) that is communicatively coupled to one or more non-transitory computer-readable media capable of storing instructions (e.g., in the form of applications or programs) that the processing circuitry may execute. The one or more non-transitory computer-readable media may be included in the data processing system 64 or communicatively coupled to the processing circuitry of the data processing system. As such, the data processing system 64 may receive data (e.g., logging data such as isotope logging data) from the analyzer 60 regarding analyzed mud gas or other gasses, and, utilizing processing circuitry that executes instructions stored on the one or more non-transitory computer-readable media, analyze the logging data to make determinations regarding the mud gas and/or or control one or more components of the drilling rig system 5, such as the analyzer 60 or the BHA 50. With that said, it should be noted that the techniques described herein as being performed by the data processing system 64 may be performed, in other embodiments, by a remotely located computing device or system that receives data, for example, from the analyzer 60. Furthermore, it should be noted that the data processing system 64 may include and/or be communicatively coupled to one or more displays. As such, the data processing system 64 may also cause data or representations of data to be displayed.

As noted above, the present disclosure relates to the characterization of fluid or gas (e.g., mud gas) samples. In particular, the presently described techniques enable the automated conversion of advanced mud gas logging data into information about fluid type, fluid properties, and fluid alterations. Specifically, δ¹³C values of gaseous alkanes can be utilized to determine thermal maturity (e.g., scaled to vitrinite reflectance equivalent (VRE)) and gas-oil ratio (GOR).

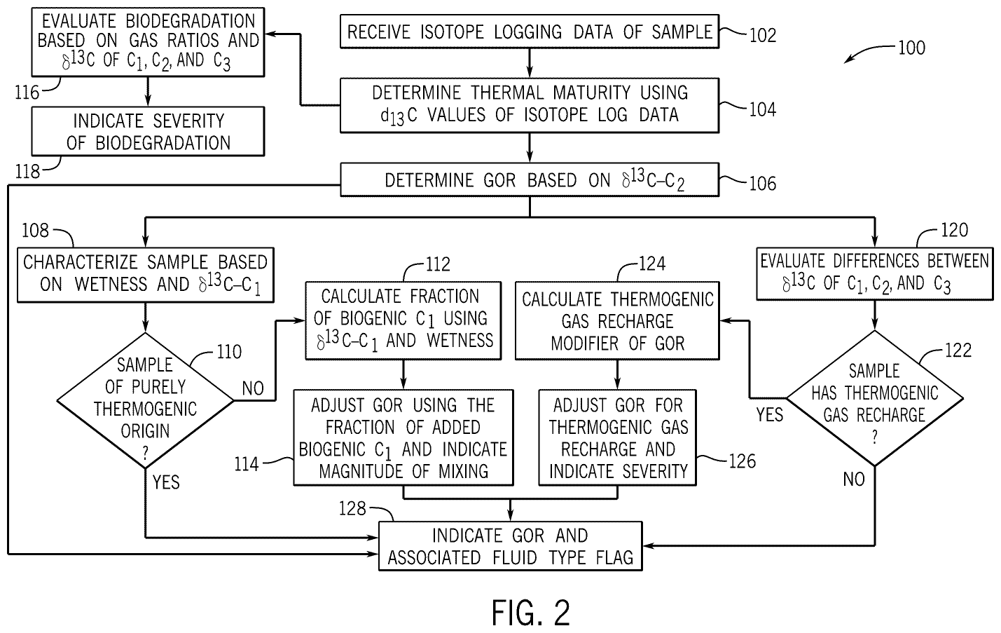

Continuing with the drawings, FIG. 2 is a flow diagram of a process 100 for characterizing subterranean formations and fluids of subterranean formations. The process 100 may be performed by processing circuitry, such as processing circuitry of the data processing system 64, executing instructions stored on a computer-readable medium. However, while operations of the process 100 are discussed below as performed by the data processing system 64, it should be noted that the process 100 may be performed by processing circuitry that is communicatively coupled to the analyzer 60 or configured to receive logging data, such as isotope logging data. Additionally, while the operations of the process 100 are discussed in one order below, it should be noted that, in other embodiments, operations of the process 100 may be performed in another order and/or omitted. Bearing this in mind, the process 100 generally includes receiving isotope logging data (process block 102) of a sample (e.g., of mud gas), determining a thermal maturity of the sample using δ¹³C (which may also be written as “d₁₃C”) values of the isotope logging data (process block 104), determining a gas-oil ratio (GOR) for the sample based on δ¹³C-C₂ (process block 106), characterizing the sample based on wetness and δ¹³C-C₁ (process block 108), determining whether the sample is of purely thermogenic origin (decision block 110), and upon determining that the sample is not of purely thermogenic origin, determining a fraction of biogenic methane using δ¹³C-C₁ and wetness (process block 112) and modifying the GOR using the fraction of added biogenic methane and indicating a magnitude of mixing of the sample (process block 114). The process 100 may also include evaluating biodegradation of the sample (process block 116) and indicating a severity of biodegradation (process block 118). Additionally, the process 100 may include evaluating differences between δ¹³C-C₁, δ¹³C-C₂, and δ¹³C-C₃ (process block 120), determining whether the sample has thermogenic gas recharge (decision block 122), and, upon determining the sample has thermogenic gas recharge, determining a thermogenic recharge modifier for the GOR (process block 124) and adjusting the GOR for thermogenic gas recharge and indicating a severity of the thermogenic gas recharge (process block 126). Additionally, the process 100 may include indicating the GOR and an associated fluid type (process block 128), which may be done after process block 106, after process block 114, after process block 126, upon determining the sample is of purely thermogenic origin (e.g., at decision block 110), or upon determining there is no thermogenic gas recharge (e.g., at decision block 122)

At process block 102, processing circuitry of the data processing system 102 may receive logging data (which may also be referred to as “log data”) of a sample. For example, the logging data may be generated by the analyzer 60 regarding mud gas (e.g., after being extracted from drilling mug by the extractor 58). More specifically, the logging data may be isotope logging data that includes δ¹³C values for one or more alkanes. For example, in one embodiment, the δ¹³C values may include values of δ¹³C-C₁ (for methane) and δ¹³C-C₂ (for ethane). In another embodiment, the δ¹³C values may additionally include δ¹³C-C₃ values (for propane). In yet another embodiment, the δ¹³C values may also include values for δ¹³C-C₄ (for butane) and δ¹³C-C₅ (for pentane). The logging data may also include data indicative of the (relative) amounts of different alkanes in a sample and/or a gas wetness ratio.

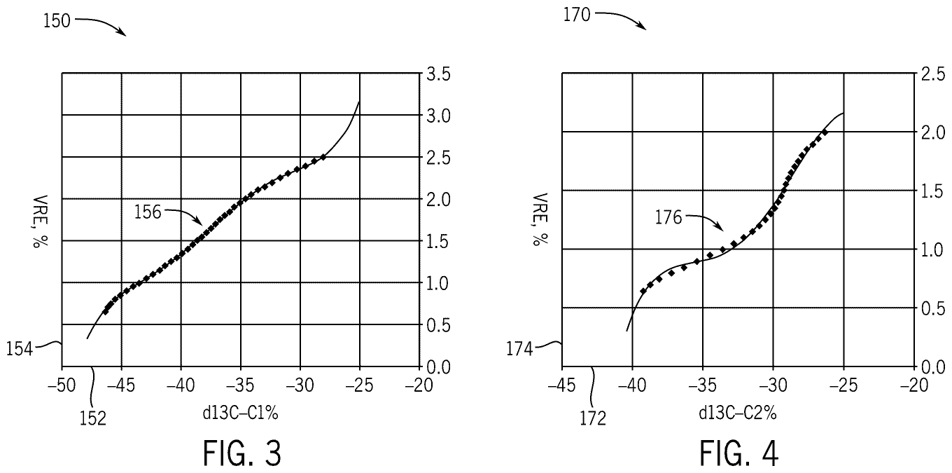

At process block 104, the data processing system may determine a thermal maturity of the sample using the δ¹³C values of the logging data receiving at process block 102. In particular, the data processing system 64 may determine a maturity for the sample based on several alkanes, such as methane, ethane, and propane. Thus, multiple thermal maturities may be determined at process block 104. Thermal maturity may be reflected by a value (e.g., a percentage) of vitrinite reflectance equivalent (VRE). The data processing system 64 may determine values of VRE for alkanes based on the δ¹³C values. More specifically, the data processing system 64 may determine VRE values by converting δ¹³C values to CRE values based on alkane-specific polynomial equations. For example, FIG. 3 is a graph 150 of δ¹³C-C₁ (as indicated by axis 152) versus VRE (indicated by axis 154). The graph 150 includes a line 156 of a polynomial equation for converting δ¹³C-C₁ to a VRE for methane. As another example, FIG. 4 is a graph 170 of δ¹³C-C₂ (as indicated by axis 172) versus VRE (indicated by axis 174), and the graph 170 includes a line 176 of a polynomial equation for converting δ¹³C-C₂ to a VRE for ethane. The polynomial equations utilized to convert δ¹³C values to VRE values may orders of three, four, or five. In other words, the highest exponent included in the equations may be three, four, or five. Additionally, the polynomials equations may be derived from works that describe VRE modeling of δ¹³C for alkanes, such as “Empirical carbon isotope/maturity relationships for gases from algal kerogens and terrigenous organic matter, based on dry, open-system pyrolysis” by U. Berner and E. Faber published in volume 24, no. 10-11 in pages 947-955 of Organic Geochemistry. Additionally, the polynomial equations may be tuned to cover the full range of natural values of δ¹³C of alkanes. For instance, in the graph 150 and for methane, VRE may be calculated from −48‰ to −25‰, which respectively correspond to the lowest measurable vitrinite reflectance (Ro) about 0.3% (e.g., the most immature source rock at the very beginning of generation hydrocarbons) and a high maturity well (e.g., dry gas). For ethane, and as depicted by the line 176 in the graph 170, the polynomial equation may return values of VRE ranging from 0.3% to 2.15% for values of δ¹³C-C₂ ranging from −40.5‰ to −25‰. Additionally, for propane, for values of δ¹³C-C₃ ranging from 0.3% to 2.25%, VRE values ranging from −36.5‰ to −24.45‰ may be determined.

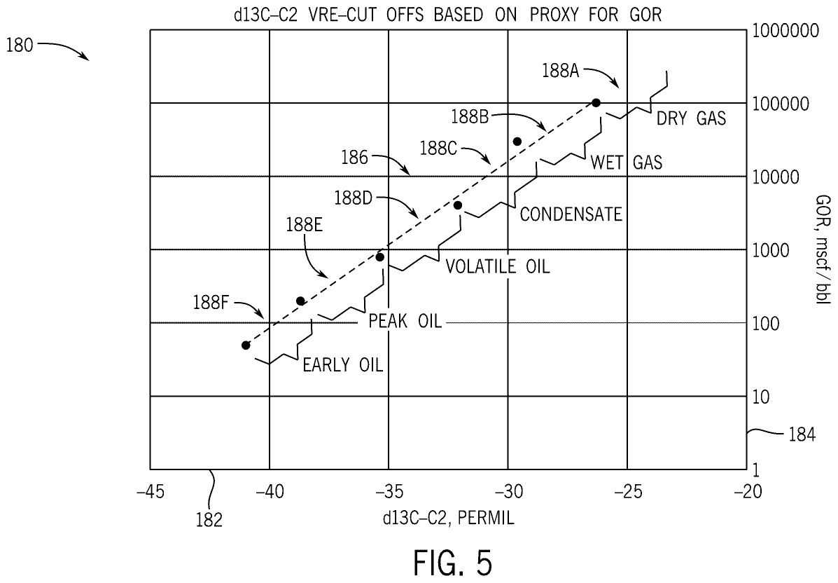

At process block 106, the data processing system 64 may determine a gas-oil ratio (GOR) for the sample based on the δ¹³C-C₂ value of the sample. To determine the GOR, the data processing system 64 may utilize an equation that converts δ¹³C-C₂ values to GOR. For instance, FIG. 5 is a graph 180 plotting δ¹³C-C₂ (as indicated by axis 182) versus GOR (as indicated by axis 184) in which line 186 is representative of an equation for converting δ¹³C-C₂ values to GOR values. The equation may be a natural exponential equation (e.g., an equation in which a constant is multiplied by e raised to an exponential value that may include, or be derived from, a δ¹³C-C₂ value). In the graph 180, the axis 184 is logarithmic, and various portions 188 (collectively referring to portions 188A-188F) of the line 186 correspond to fluid types. The portions 188 may include portion 188A (corresponding to dry gas), portion 188B (corresponding to wet gas), portion 188C (corresponding to condensate), portion 188D (corresponding to co-genetic to volatile oil), portion 188E (corresponding to co-genetic to peak oil), and portion 188F (corresponding to co-genetic to early oil). Accordingly, the data processing system 64 may determine a GOR for the sample based on a δ¹³C-C₂ value of the sample.

Referring back to FIG. 2 and continuing the discussion of the process 100, at process block 108, the data processing system 64 may characterize the sample based on wetness (e.g., gas wetness ratio) and δ¹³C-C₁. The data processing system 64 may receive the gas wetness ratio as part of the logging data received at process block 102 or determine the gas wetness ratio from values of the received logging data indicative of the amounts (e.g., in parts per million (PPM)) of different alkanes in the sample. For example, the data processing system 64 may determine the gas wetness ratio by determining a first sum of the amounts of ethane, propane, butane, and pentane in a sample, determining a second sum of the amounts of methane, ethane, propane, butane, and pentane in the sample, and dividing the first sum by the second sum (and optionally multiplying the quotient by one-hundred to generate a value that is a percentage).

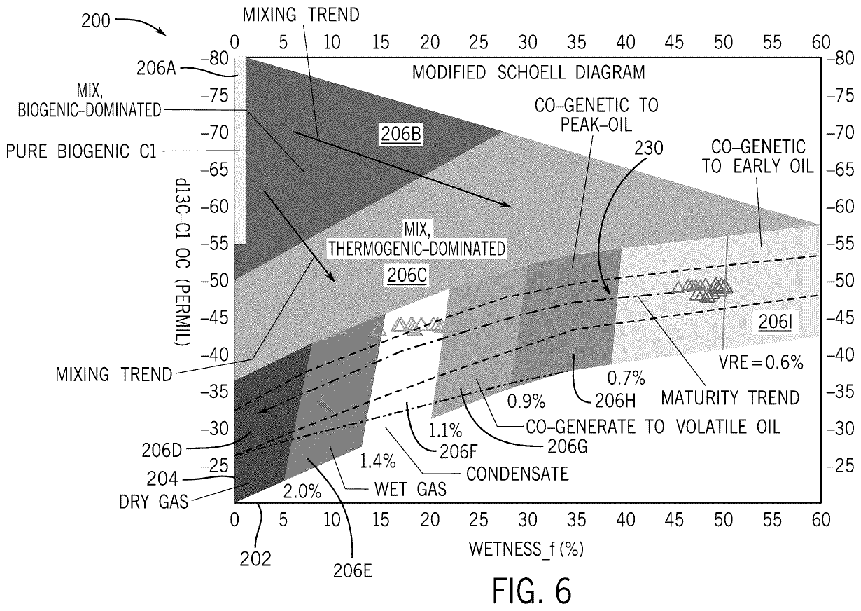

To characterize the sample based on the wetness and δ¹³C-C₁, the data processing system 64 may determine a range or region (e.g., as defined by a graph such as graph 200 of FIG. 6 into which a given wetness and δ¹³C-C₁ falls. In particular, FIG. 6 is a diagram or graph 200 plotting wetness (e.g., gas wetness ratio, as indicated by the axis 202) versus δ¹³C-C₁ (as indicated by axis 204). The graph 200 also includes regions 206 (collectively referring to regions 206A-206I). The regions 206, which correspond to fluid types, may include region 206A (corresponding to pure biogenic methane), region 206B (corresponding to a biogenic-dominated mix), region 206C (corresponding to a thermogenic-dominated mix), region 206D (corresponding to dry gas), region 206E (corresponding to wet gas), region 206F (corresponding to condensate), region 206G (corresponding to co-genetic to volatile oil), region 206H (corresponding to co-genetic to peak oil), and region 206I (corresponding to co-genetic to early oil). Some of the regions 206 may correspond to the portions 186 of the graph 180 of FIG. 5. For example, region 206D corresponds to portion 188A, region 206E corresponds to portion 188B, region 206F corresponds to portion 188C, region 206G corresponds to portion 188D, region 206H corresponds to portion 188E, and region 206I corresponds to portion 188F. Thus, the regions 206 correspond to fluid types. The graph 200 or data representative or indicative of the graph 200 may be stored in a computer-readable medium and utilized by the data processing system 64 to perform the operations of process block 206. Indeed, to characterize a sample, the data processing system 64 may determine into which of the regions 206 a sample falls based on the gas wetness ratio of the sample and the value of δ¹³C-C₁ of the sample.

Returning to FIG. 2 and the discussion of the process 100, at decision block 110, the data processing system 64 may determine whether the sample is of purely thermogenic origin, for example, based on the characterization performed at process block 108. More particularly, and referring to FIG. 6, the data processing system 64 may determine the sample is of purely thermogenic origin upon determining that the sample falls in the region 206D, the region 206E, the region 206F, the region 206G, the region 206H, or the region 206I of the graph 200. In other words, if a point were added to the graph 200 based on the gas wetness ratio and the δ¹³C-C₁ of the sample, and the point were located in one of the regions 206D-206I, the data processing system 64 may determine the sample is of purely thermogenic origin. Conversely, the data processing system 64 may determine the sample is not of purely thermogenic origin upon determining the point is located outside of one of the regions 206D-206I. For example, if the point were to be located inside of the region 206A, the region 206B, or the region 206C, the data processing system 64 may determine the sample is not of purely thermogenic origin. As discussed below, the GOR for the sample (e.g., as determined at process block 106) may be modified based on determining that the sample is not of purely thermogenic origin.

Returning to FIG. 2 and the discussion of the process 100, if at decision block 110 the data processing system 64 determines the sample is not of purely thermogenic origin, at process block 112, the data processing system 64 may determine a fraction of the sample that is biogenic methane based on δ¹³C-C₁ as well as the gas wetness ratio value of the sample. To determine the fraction of biogenic methane, the data processing system 64 may determine a value that is correlated to a distance from a trend line 230 of the graph 200 that a point for the sample would be when plotted on the graph 200. More specifically, the fraction of biogenic methane may be determined by determining a quotient of a difference divided by a sum in which the difference is determined by subtracting δ¹³C-C₁ from a value, and the sum is determined by adding the value to a constant. The constant may be an integer (e.g., a value between fifty and one-hundred, inclusive), and the value may be determined based on the gas wetness ratio of the sample. More specifically, the value may be determined using a second-degree polynomial equation in which a first constant (e.g., a value between 0.001 and 0.01, inclusive) is multiplied is square of the gas wetness ratio to obtain a first value. From the first value, a second value is subtracted to obtain a third value. The second value may be determined by multiplying the gas wetness ratio by a second constant (e.g., a value between zero and one, inclusive). Lastly, a third constant (e.g., a value between ten and fifty, inclusive) is subtracted from the third value to obtain the fraction of biogenic methane.

At process block 114, the data processing system 64 may modify the GOR (e.g., as determined at process block 106) based on the fraction of biogenic methane determined at process block 112. In one embodiment, to modify the GOR, the data processing system 64 may multiply the GOR (as determined at process block 106) by a value that is the reciprocal of the difference between one and fraction of biogenic methane (determined at process block 112). As such, the data processing system 64 may modify the GOR determined to account for the sample not being purely thermogenic origin.

At process block 114, the data processing system 64 may additionally indicate a magnitude of mixing based on the fraction of the biogenic methane determined at process block 112. For example, the data processing system 64 may cause a visual representation indicative of the degree of mixing in the sample to be displayed. Such a visual representation may be a graph or included in a graph.

Referring back to FIG. 2 and continuing with the discussion of the process 100, at process block 116, the data processing system 64 may evaluate biodegradation of the sample, for example, based on the thermal maturity of the sample (e.g., as determined at process block 104). Biodegradation can be a critical alteration of the original petroleum fluid, oil, or gas, leading to overall compositional changes that impact fluid properties, such as viscosity and mobility. Biodegradation also adds its product to the fluid, biogenic methane, which increases GOR. As such, the data processing system 64 may evaluate biodegradation to determine an extent or severity of biodegradation (if any). In one embodiment, the data processing system 64 may evaluate biodegradation of the sample by determining a number of conditions present. The conditions may include, for example: 1) a difference between the VRE determined based on δ¹³C-C₃ and the VRE determined based on δ¹³C-C₂ exceeding a threshold value (e.g., a value between 0 and 0.5, inclusive); 2) the ratio of the quantities of isobutane to butane in the sample (e.g., as indicated in the received logging data) is greater than one; 3) the ratio of the quantities of isopentane to pentane in the sample (e.g., as indicated in the received logging data) is greater than one; 4a) the value of δ¹³C-C₃ being less than −49 or 4b) if the value of δ¹³C-C₃ is greater than −49, the difference of the VRE determined based on δ¹³C-C₂ and VRE determined based on δ¹³C-C₁ exceeding a threshold value (which may be the same value as the threshold used in the first condition); 5) the value of δ¹³C-C₃ exceeding −22; and 6) a ratio exceeding another threshold value (e.g., between 0 and 0.5, inclusive), in which the ratio is a ratio of a first value (e.g., a ratio of the quantities of ethane to propane in the sample (e.g., as indicated by the received logging data)) to a second value, which may be a ratio of the quantities of ethane to isobutane (e.g., as indicated in the received logging data) in the sample.

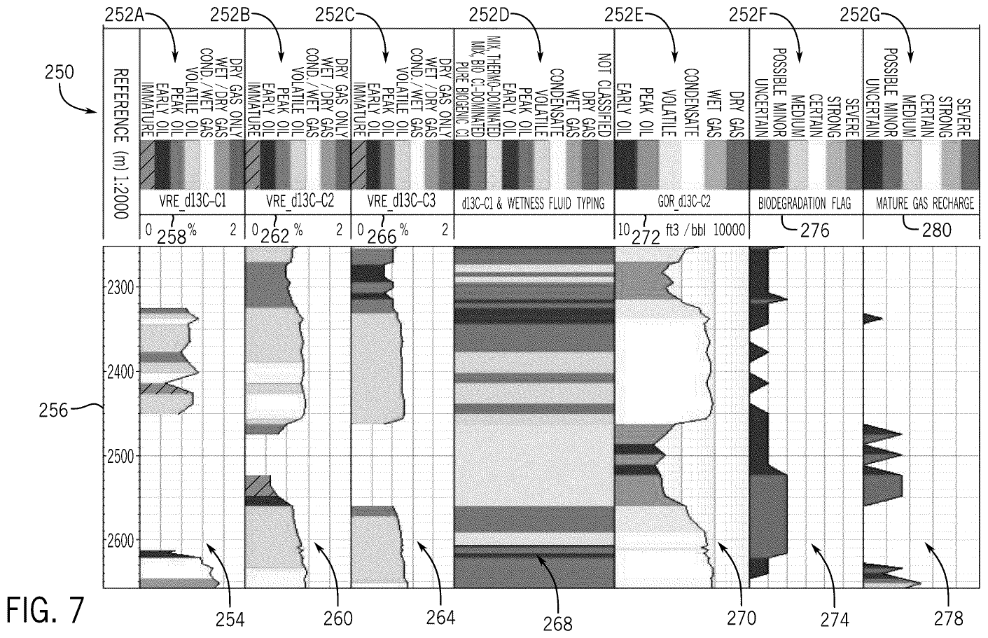

At process block 118, the data analysis system 64 may indicate a severity of biodegradation, for example, by causing a graphical representation of the number of conditions discussed above with respect to process block 116 that are present (and/or an indication as to which conditions are present). For example, FIG. 7 is a chart 250 that includes sections 252 (collectively referring to sections 252A-252G). Section 252A includes a graph 254 having axis 256 (indicative of a reference point, such as a point indicative of when mud gas was analyzed or from where (e.g., a depth) mud gas was collected and analyzed)) and axis 258, which is indicative of the VRE determined at the reference point as determined based on δ¹³C-C₁. Various portions of the graph 254 are indicative of the thermal maturity at various reference points along the axis 256 (e.g., for which samples were analyzed), as indicated by the various shading or hatching. Section 252B includes a graph 260 having axis 256 and axis 262 (indicative of the VRE determined at the reference point as determined based on δ¹³C-C₂) that, like the graph 254, is indicative of the thermal maturity at various reference points along the axis 256. Somewhat similarly, Section 252C includes a graph 264 having the axis 256 and axis 266 (indicative of the VRE determined at the reference point as determined based on δ¹³C-C₃) that, like the graph 254, is indicative of the thermal maturity at various reference points along the axis 256. Section 252D includes a graph 268 indicative of a thermal maturity for a given reference points along the axis 256 as based on the sections 206 of the graph 200 of FIG. 6. In other words, the graph 268 is indicative of in which section 206 of the graph 200 a point corresponding to a given reference point (along the axis 256) would be. Section 252E includes a graph 270 indicative of GOR (as indicated by axis 272) for a given reference point (as indicated by the axis 256) as well as the thermal maturity for the given reference point. The GOR indicated in the graph 270 may be the GOR determined at process block 110, process block 112, or (as discussed below) process block 124. Section 252F includes a graph 274 that has the axis 256 and axis 276, which is indicative of severity of biodegradation (which is also indicated by shading or hatching). Section 252G includes a graph 278 that is indicative of thermogenic gas recharge (e.g., mature gas recharge) as indicated along axis 280 (and by shading or hatching) for particular reference points (indicated along the axis 256). Thus, to indicate a severity of biodegradation, in process block 118, the data analysis system 64 may generate and cause the graph 274 to be displayed. In some embodiments, the operations of process block 116 may be performed contemporaneously with the operations associated with process block 122 or process 124, which are discussed below.

Returning to FIG. 2 and the discussion of the process 100, at process block 120, the data processing system 64 may evaluate differences between isotope ratios for methane, ethane, and propane, for example, to evaluate thermogenic gas recharge, which may also be referred to as “mature gas recharge.” In many reservoirs, less mature fluid (e.g., hydrocarbons) is followed by a more mature fluid, often gas, as a consequence of increased burial depth and hence temperature of underlying source rocks, due to accumulating sediments in a basin, (e.g., deltaic systems). Such late arriving more thermally mature gas bears “heavier” ¹³C-enriched isotopic signatures. That will cause appearance of higher VRE of lighter gases, more concentrated in mature gas, when mixed with lower maturity fluid, such as black oil-associated gas. As a result, an expected pattern in the mixture is that the VRE determined based on δ¹³C-C₁ (VRE_{C1}) will be greater than the VRE determined based on δ¹³C-C₂ (VRE_{C2}), which will be greater than the VRE determined based on δ¹³C-C₁ (VRE_{C3}). The data processing system 64 may take into account the amount of excess VRE of all three pairs of C₁, C₂, and C₃ (e.g., caused due to thermogenic gas recharge).

Indeed, to evaluate thermogenic gas recharge, at process block 120, the data processing system 64 may determine whether which of the following conditions are present: 1a) a difference between the VRE determined based on δ¹³C-C₁ and the VRE determined based on δ¹³C-C₂ exceeding or being equal to a first threshold value (e.g., a value between 0 and 0.5, inclusive), which may be indicative of a relatively higher degree of thermogenic gas recharge (compared to 1b); 1b) when the difference determined at 1a is less than the first threshold, the difference exceeding a second threshold that is less than the first threshold value; 2a) a difference between the VRE determined based on δ¹³C-C₁ and the VRE determined based on δ¹³C-C₃ exceeding or being equal to a first threshold value (e.g., the same threshold value used in 1a), which may be indicative of a relatively higher degree of thermogenic gas recharge (compared to 2b); 2b) when the difference determined at 2a is less than the first threshold, the difference exceeding a second threshold that is less than the first threshold value (e.g., the same second threshold used above in 1b); 3a) a difference between the VRE determined based on δ¹³C-C₂ and the VRE determined based on δ¹³C-C₃ exceeding or being equal to a first threshold value (e.g., the same threshold value used in 1a), which may be indicative of a relatively higher degree of thermogenic gas recharge (compared to 3b); and 3b) when the difference determined at 3a is less than the first threshold, the difference exceeding a second threshold that is less than the first threshold value (e.g., the same second threshold used above in 1b). Each time the first threshold is exceeded (or equaled) by a determined difference, the data processing system 64 may assign a degree or severity of mature gas recharge that is greater than (e.g., double) a degree or severity assigned when the first threshold is not exceed (or equaled) but the second threshold is exceeded. When either threshold is exceeded (or, in the case of the first threshold, equaled), the data processing system 64 may determine there is thermogenic gas recharge.

At decision block 122, the data processing system 64 may determine whether the sample has thermogenic gas recharge based on the differences evaluated in process block 120. For example, when the first threshold or the second threshold described in the preceding paragraph is exceeded (or, in the case of the first threshold, equaled), the data processing system 64 may determine there is thermogenic gas recharge.

If at decision block 122 the data processing system 64 determines to adjust the GOR (e.g., based on determining there is thermogenic gas recharge), at process block 126, the data processing system 64 may determine a thermogenic gas recharge modifier to the GOR (e.g., as determined at process block 106 or at process block 114) based on the degree or severity of thermogenic gas recharge. The thermogenic gas recharge modifier may be determined as a quotient of an integer value representative of the determined severity of thermogenic gas recharge divided by an integer representative of a maximum possible severity of thermogenic gas recharge. The integer representative of the maximum possible severity of thermogenic gas recharge, which may occur when the first threshold (described two paragraphs above) is exceeded or equaled by each of the three differences described two paragraphs above.

At process block 126, the data processing system 64 may modify the GOR based on the thermogenic gas recharge modifier. For example, the data processing system 64 may multiply the determined GOR (e.g., the GOR determined at process block 106 or the GOR as modified at process block 114, if applicable) by a sum of one and the thermogenic gas recharge modifier. As such, the GOR may be modified to account for the degree or severity of thermogenic gas recharge in the sample. Additionally, also at process block 126, the data processing system 64 may indicate the severity of thermogenic gas recharge. More specifically, the data processing system 64 may cause the degree of thermogenic gas recharge (or a representation thereof) to be displayed, for example, as a value or in the form of the graph 278.

At process block 128, the data processing system 128 may indicate the GOR as well as an associated fluid type of the sample. For example, the data processing system 64 may cause the GOR (or a representation of the GOR (as determined at process block 106 or as modified at process block 114 or process block 126), which may be included in the graph 270) to be displayed, either alone or with an indication of a fluid type of the sample (e.g., a value or the graph 268). It should also be noted that the data processing system 64 may cause the chart 250 or any portion 252 thereof to be displayed when performing the operations of process block 128. The operations of process block 128 may be performed after determining the GOR (e.g., at process block 106), in response to determining the sample is of purely thermogenic origin (e.g., at decision block 110), determining the sample does not have thermogenic gas recharge (e.g., at decision block 122), or after modifying the GOR (e.g., at process block 114 or process block 126).

In other embodiments, the process 100 may include operations additional to those described above. For example, in response to characterizing a sample as being of purely thermogenic origin (or not of purely thermogenic origin), determining there is or may be biodegradation, determining there is or may be thermogenic gas recharge, or any combination thereof, the data processing system 64 may alter a rate of sampling, cause the position of the BHA 50 to be altered, cause a rate at which the BHA 50 (or a portion thereof) traverses the subterranean geological formation 32 to be altered, or any combination thereof. For instance, in one embodiment, the data processing system 64 may decrease a rate of sampling of mud gas in response to determining a sample is of purely thermogenic origin or in response to determining the sample is not of purely thermogenic origin. In another embodiment, the data processing system 64 may increase a rate of sampling of mud gas in response to determining a sample is of purely thermogenic origin or in response to determining the sample is not of purely thermogenic origin. As another example, the data processing system 64 may decrease a rate of sampling of mud gas in response to determining there is biodegradation and/or in response to determining there is thermogenic gas recharge. As yet another example, the data processing system 64 may increase a rate of sampling of mud gas in response to determining there is biodegradation and/or in response to determining there is thermogenic gas recharge.

In another embodiment, the data processing system 64 may cause the position of the BHA 50 to be raised or lowered, cause a rate at which the BHA 50 traverses the subterranean geological formation 32 to be increased or decreased, or both in response to determining a sample is of purely thermogenic origin or in response to determining the sample is not of purely thermogenic origin. In yet another embodiment, the data processing system 64 may cause the position of the BHA 50 to be raised or lowered, cause a rate at which the BHA 50 traverses the subterranean geological formation 32 to be increased or decreased, or both in response to determining there is biodegradation and/or in response to determining there is thermogenic gas recharge. As yet another example, the data processing system 64 may cause the position of the BHA 50 to be raised or lowered, cause a rate at which the BHA 50 traverses the subterranean geological formation 32 to be increased or decreased, or both in response to determining there is biodegradation and/or in response to determining there is thermogenic gas recharge.

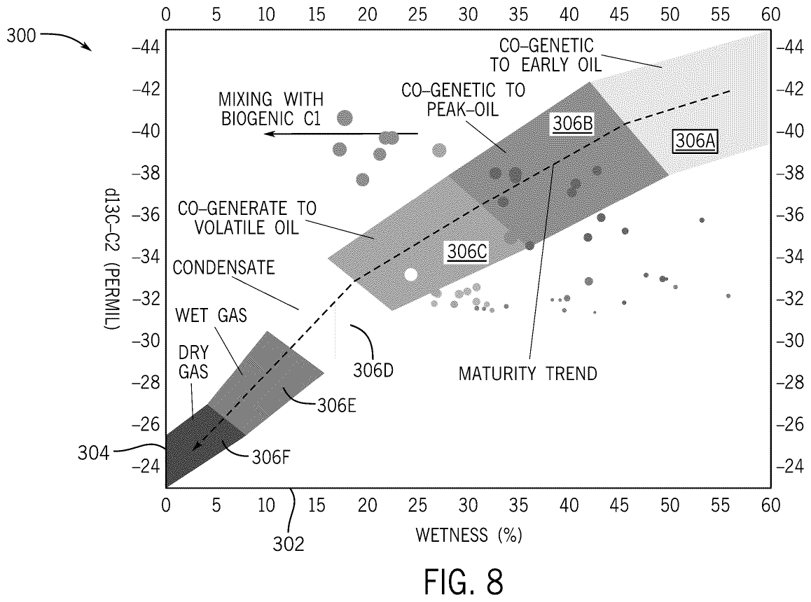

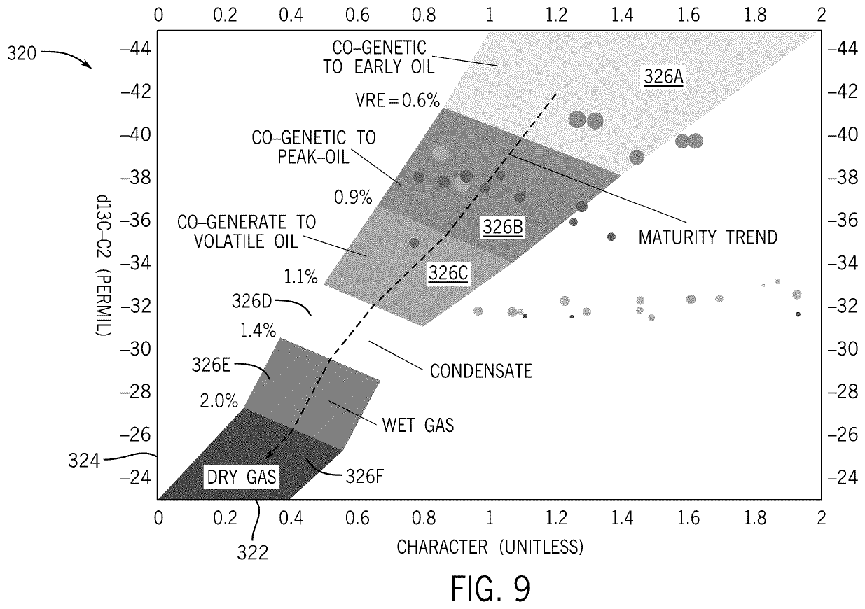

As discussed below with respect to FIG. 8 and FIG. 9, the techniques of the present application may also enable characterization (e.g., of fluid maturity) using δ¹³C-C₂ along with two different ratios as proxies for thermal maturity. In particular, FIG. 8 is a graph 300 plotting gas wetness ratio (as indicated by axis 302) against δ¹³C-C₂ (as indicated by axis 304). The graph 300 also includes regions 306 (collectively referring to regions 306A-306F) corresponding to regions 206D-206I of the graph 200 of FIG. 5. For example, portion 306A corresponds to region 206D, portion 306B corresponds to region 206E, portion 306C corresponds to region 206F, portion 306D corresponds to region 206G, portion 306E corresponds to region 206H, and portion 306F corresponds to region 206I. FIG. 9 is a graph 320 plotting gas character (as indicated by axis 322) against δ¹³C-C₂ (as indicated by axis 324). The data analysis system 64 may determine a value for gas character by dividing a sum of the amounts of butane and pentane in a sample by the amount of propane in the sample. The graph 320 also includes regions 326 (collectively referring to regions 326A-326F) corresponding to regions 206D-206I of the graph 200 of FIG. 5. For example, portion 326A corresponds to region 206D, portion 326B corresponds to region 206E, portion 326C corresponds to region 206F, portion 326D corresponds to region 206G, portion 326E corresponds to region 206H, and portion 326F corresponds to region 206I. The data processing system 64 may characterize samples based on in which of the regions 306, 236 data points for the samples are located.

In one embodiment, a system includes processing circuitry and a non-transitory, computer-readable medium including instructions that, when executed by the processing circuitry, cause the processing circuitry to receive logging data regarding a fluid. The logging data is indicative of a plurality of isotope ratios of a plurality of alkanes of the fluid. The instructions, when executed, also cause the processing circuitry to determine a thermal maturity of the fluid, based on at least a first isotope ratio of the plurality of isotope ratios of the logging data corresponding to a first alkane of the plurality of alkanes. Additionally, when executed, the instructions cause the processing circuitry to determine a gas-oil ratio (GOR) of the fluid based on at least a second isotope ratio of the plurality of isotope ratios corresponding to a second alkane of the plurality of alkanes. Moreover, when executed, the instructions cause display of the thermal maturity of the fluid and the GOR of the fluid.

The logging data may include a plurality of amounts, wherein each respective amount of the plurality of amounts corresponds to a respective alkane of the plurality of alkanes. Furthermore, when executed, the instructions may further cause the processing circuitry to determine a gas wetness ratio of the fluid based on at least a portion of the plurality of amounts and determine the thermal maturity of the fluid based on the gas wetness ratio.

The instructions, when executed, may further cause the processing circuitry to determine the GOR based at least on the second isotope ratio and determine whether the fluid is of purely thermogenic origin based on the thermal maturity of the fluid and the gas wetness ratio. When executed, the instructions may also cause the processing circuitry to generate a modified GOR by modifying the GOR based at least on the first isotope ratio and the gas wetness ratio in response to determining the fluid is not of purely thermogenic origin.

When executed, the instructions may additionally cause the processing circuitry to determine a degree of thermogenic gas recharge of the fluid and modify the GOR or the modified GOR based on the degree of the thermogenic gas recharge of the fluid.

The first alkane may be methane, and the second alkane may be ethane. Additionally, the thermal maturity comprises a vitrinite reflectance equivalent (VRE) value. Furthermore, when executed, the instructions may further cause the processing circuitry to cause a rate of sampling of the fluid to be increased or decreased based on the thermal maturity of the fluid. Moreover, the system may include a drilling system that is communicatively coupled to the processing circuitry. When executed, the instructions may cause the processing circuitry to adjust the drilling system based on the thermal maturity of the fluid.

In another embodiment, a non-transitory, computer-readable medium includes instructions that, when executed by processing circuitry, cause the processing circuitry to receive logging data regarding a fluid. The logging data is indicative of a plurality of isotope ratios of a plurality of alkanes of the fluid, and the logging data includes a plurality of amounts. Each respective amount of the plurality of amounts corresponds to a respective alkane of the plurality of alkanes. When executed, the instructions also cause the processing circuitry to determine a thermal maturity of the fluid based on at least a first isotope ratio of the plurality of isotope ratios of the logging data corresponding to a first alkane of the plurality of alkanes. Additionally, the instructions, when executed, cause the processing circuitry to determine a gas-oil ratio (GOR) of the fluid based on at least a second isotope ratio of the plurality of isotope ratios corresponding to a second alkane of the plurality of alkanes. When executed, in the instructions also cause the processing circuitry to determine a gas wetness ratio of the fluid based on at least a portion of the plurality of amounts, determine a fluid typing of the fluid based on the gas wetness ratio and the first isotope ratio, and cause display of the thermal maturity of the fluid, the fluid typing of the fluid, and the GOR of the fluid.

The fluid typing may include one of pure biogenic methane, a biogenic-dominated mix, a thermogenic-dominated mix, dry gas, wet gas, condensate, volatile oil, peak oil, or early oil. Additionally, the thermal maturity of the fluid may include a plurality of vitrinite reflectance equivalent (VRE) values. The plurality of VRE values may include a first VRE determined based on at least on the first isotope ratio, a second VRE determined based on at least on the second isotope ratio, and a third VRE determined based on at least a third isotope ratio of the plurality of isotope ratios corresponding to a third alkane of the plurality of alkanes.

When executed, the instructions may cause the processing circuitry to determine a degree of biodegradation of the fluid based on the logging data, determine a degree of thermogenic gas recharge of the fluid, and modify the GOR based on the degree of the thermogenic gas recharge of the fluid. Additionally, the instructions, when executed may cause the processing circuitry to cause display of a first visual indication of the plurality of VRE values, a second visual indication of the degree of biodegradation, and a third visual indication of the degree of thermogenic gas recharge. When executed, the instructions may cause the processing circuitry to determine the degree of biodegradation of the fluid based on determining whether a difference between the second VRE and the third VRE exceeds a first threshold value, whether a first ratio exceeds a second threshold value (in which the first ratio is a ratio of a first amount of the plurality of amounts corresponding to a fourth alkane of the plurality of alkanes to a second amount of the plurality of amounts corresponding to a fifth alkane of the plurality of alkanes), whether a second ratio exceeds a third threshold value (in which the second ratio is a ratio of a third amount of the plurality of amounts corresponding to a sixth alkane of the plurality of alkanes to a fourth amount of the plurality of amounts corresponding to a seventh alkane of the plurality of alkanes), whether the third isotope ratio is less than a fourth threshold value (or, if the value of the third isotope ratio is greater than the fourth threshold, whether a second difference of the second VRE value and the first VRE value exceeds a fifth threshold value), whether the third isotope ratio exceeds a sixth threshold, whether a third ratio exceeds a seventh threshold value (in which the third ratio is a ratio of a fourth ratio to a fifth ratio, with the fourth ratio being a ratio of a fifth amount of the plurality of amounts corresponding to the second alkane to a sixth amount of the plurality of amounts corresponding to the third alkane, and the fifth ratio being a ratio of the fifth amount to the first amount), or a combination thereof.

In yet another embodiment, a computer-implemented method includes receiving, via processing circuitry, logging data regarding a fluid, wherein the logging data is indicative of a plurality of isotope ratios of a plurality of alkanes of the fluid. The computer-implemented method also includes determining a thermal maturity of the fluid via the processing circuitry and based on at least a first isotope ratio of the plurality of isotope ratios of the logging data corresponding to a first alkane of the plurality of alkanes. Additionally, the computer-implemented method includes determining a gas-oil ratio (GOR) of the fluid via the processing circuitry and based on at least a second isotope ratio of the plurality of isotope ratios corresponding to a second alkane of the plurality of alkanes. Moreover, the computer-implemented method includes causing, via processing circuitry, display of the thermal maturity of the fluid and the GOR of the fluid.

The computer-implemented method may include determining a plurality of vitrinite reflectance equivalent (VRE) values based on the plurality of isotope ratios. The plurality of VRE values includes a first VRE determined based on at least on the first isotope ratio, a second VRE determined based on at least on the second isotope ratio, and a third VRE determined based on at least a third isotope ratio of the plurality of isotope ratios corresponding to a third alkane of the plurality of alkanes. The first alkane may be methane, the second alkane may be ethane, and the third alkane may be propane.

The computer-implemented method may also include causing, via the processing circuitry, a drilling system to be adjusted based on the thermal maturity of the fluid, causing, via the processing circuitry, a rate of sampling of the fluid to be increased or decreased based on the thermal maturity of the fluid, or both.

Accordingly, the techniques disclosed herein enable hydrocarbon-containing fluids, such as mud gas, to be analyzed and characterized, for example, to log subterranean formations or as part of logging subterranean formations. Indeed, as described above, logging data (e.g., advanced mud gas logging data) including data regarding isotope ratios of hydrocarbons may be utilized to determine fluid properties (e.g., fluid maturity, gas-oil ratio), biodegradation, and recharge. Furthermore, although the examples described above are illustrated for wellbores on the land, similar method may be applied to any acquisition configuration.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principals of the disclosure and its practical applications, to thereby enable others skilled in the art to best utilize the disclosure and various embodiments with various modifications as are suited to the particular use contemplated.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A system, comprising:
processing circuitry; and
a non-transitory, computer-readable medium comprising instructions that, when executed by the processing circuitry, cause the processing circuitry to:
receive logging data regarding a fluid, wherein the logging data is indicative of a plurality of isotope ratios of a plurality of alkanes of the fluid;
determine, based on at least a first isotope ratio of the plurality of isotope ratios of the logging data corresponding to a first alkane of the plurality of alkanes, a thermal maturity of the fluid;
determine, based on at least a second isotope ratio of the plurality of isotope ratios corresponding to a second alkane of the plurality of alkanes, a gas-oil ratio (GOR) of the fluid; and
cause display of the thermal maturity of the fluid and the GOR of the fluid.

2. The system of claim 1, wherein:
the logging data comprises a plurality of amounts, wherein each respective amount of the plurality of amounts corresponds to a respective alkane of the plurality of alkanes; and
the instructions, when executed, further cause the processing circuitry to:
determine a gas wetness ratio of the fluid based on at least a portion of the plurality of amounts; and
determine the thermal maturity of the fluid based on the gas wetness ratio.

3. The system of claim 2, wherein the instructions, when executed, further cause the processing circuitry to:
determine the GOR based at least on the second isotope ratio;
determine, based on the thermal maturity of the fluid and the gas wetness ratio, whether the fluid is of purely thermogenic origin; and
in response to determining the fluid is not of purely thermogenic origin, generate a modified GOR by modifying the GOR based at least on the first isotope ratio and the gas wetness ratio.

4. The system of claim 3, wherein the instructions, when executed, further cause the processing circuitry to:
determine a degree of thermogenic gas recharge of the fluid; and
modify the GOR or the modified GOR based on the degree of the thermogenic gas recharge of the fluid.

5. The system of claim 3, wherein:
the first alkane comprises methane; and
the second alkane comprises ethane.

6. The system of claim 1, wherein the thermal maturity comprises a vitrinite reflectance equivalent (VRE) value.

7. The system of claim 1, comprising a drilling system communicatively coupled to the processing circuitry, wherein the instructions, when executed, cause the processing circuitry to adjust the drilling system based on the thermal maturity of the fluid.

8. The system of claim 1, wherein the instructions, when executed, further cause the processing circuitry to cause a rate of sampling of the fluid to be increased or decreased based on the thermal maturity of the fluid.

9. A non-transitory, computer-readable medium comprising instructions that, when executed by processing circuitry, cause the processing circuitry to:
receive logging data regarding a fluid, wherein the logging data:
is indicative of a plurality of isotope ratios of a plurality of alkanes of the fluid; and
comprises a plurality of amounts, wherein each respective amount of the plurality of amounts corresponds to a respective alkane of the plurality of alkanes;
determine, based on at least a first isotope ratio of the plurality of isotope ratios of the logging data corresponding to a first alkane of the plurality of alkanes, a thermal maturity of the fluid;
determine, based on at least a second isotope ratio of the plurality of isotope ratios corresponding to a second alkane of the plurality of alkanes, a gas-oil ratio (GOR) of the fluid;
determine a gas wetness ratio of the fluid based on at least a portion of the plurality of amounts;
determine a fluid typing of the fluid based on the gas wetness ratio and the first isotope ratio; and
cause display of the thermal maturity of the fluid, the fluid typing of the fluid, and the GOR of the fluid.

10. The non-transitory, computer-readable medium of claim 9, wherein the fluid typing comprises one of pure biogenic methane, a biogenic-dominated mix, a thermogenic-dominated mix, dry gas, wet gas, condensate, volatile oil, peak oil, or early oil.

11. The non-transitory, computer-readable medium of claim 9, wherein the thermal maturity of the fluid comprises a plurality of vitrinite reflectance equivalent (VRE) values.

12. The non-transitory, computer-readable medium of claim 11, wherein the plurality of VRE values comprises:
a first VRE determined based on at least on the first isotope ratio;
a second VRE determined based on at least on the second isotope ratio; and
a third VRE determined based on at least a third isotope ratio of the plurality of isotope ratios corresponding to a third alkane of the plurality of alkanes.

13. The non-transitory, computer-readable medium of claim 12, wherein the instructions, when executed, cause the processing circuitry to determine a degree of biodegradation of the fluid based on the logging data.

14. The non-transitory, computer-readable medium of claim 13, wherein the instructions, when executed, cause the processing circuitry to:
determine a degree of thermogenic gas recharge of the fluid; and
modify the GOR based on the degree of the thermogenic gas recharge of the fluid.

15. The non-transitory, computer-readable medium of claim 14, wherein the instructions, when executed, cause the processing circuitry to cause display of:
a first visual indication of the plurality of VRE values;
a second visual indication of the degree of biodegradation; and
a third visual indication of the degree of thermogenic gas recharge.

16. The non-transitory, computer-readable medium of claim 13, wherein the instructions, when executed, cause the processing circuitry to determine the degree of biodegradation.

17. A computer-implemented method, comprising:
receiving, via processing circuitry, logging data regarding a fluid, wherein the logging data is indicative of a plurality of isotope ratios of a plurality of alkanes of the fluid;
determining, via the processing circuitry and based on at least a first isotope ratio of the plurality of isotope ratios of the logging data corresponding to a first alkane of the plurality of alkanes, a thermal maturity of the fluid;
determining, via the processing circuitry and based on at least a second isotope ratio of the plurality of isotope ratios corresponding to a second alkane of the plurality of alkanes, a gas-oil ratio (GOR) of the fluid; and
causing, via processing circuitry, display of the thermal maturity of the fluid and the GOR of the fluid.

18. The computer-implemented method of claim 17, comprising determining a plurality of vitrinite reflectance equivalent (VRE) values based on the plurality of isotope ratios, wherein the plurality of VRE values comprises:
a first VRE determined based on at least on the first isotope ratio;
a second VRE determined based on at least on the second isotope ratio; and
a third VRE determined based on at least a third isotope ratio of the plurality of isotope ratios corresponding to a third alkane of the plurality of alkanes.

19. The computer-implemented method of claim 18, wherein:
the first alkane comprises methane;
the second alkane comprises ethane; and
the third alkane comprises propane.

20. The computer-implemented method of claim 17, comprising:
causing, via the processing circuitry, a drilling system to be adjusted based on the thermal maturity of the fluid;
causing, via the processing circuitry, a rate of sampling of the fluid to be increased or decreased based on the thermal maturity of the fluid; or
both.
